(19) United States
(12) Patent Application Publication
(10) Pub. No.: US 2026/0183118 A1
(43) Pub. Date: Jul. 2, 2026

(54) SUPERIOR APPROACH STEMLESS IMPLANT

(71) Applicant: Biomet Manufacturing, LLC, Warsaw, IN (US)

(72) Inventor: Nathan A. Winslow, Scottsdale, AZ (US)

(21) Appl. No.: 19/131,822

(22) PCT Filed: Nov. 13, 2023
(86) PCT No.: PCT/US2023/079527

§ 371 Date: May 21, 2025

Related U.S. Application Data
(60) Provisional application No. 63/428,284, filed on Nov. 28, 2022.

(51) Int. Cl. A61F 2/40; A61F 2/30

(52) U.S. Cl. CPC A61F 2/4014; A61F 2/30771; A61F 2002/30884; A61F 2002/4051

(57) ABSTRACT

A system configured to be implanted into a patient can include a first implantable component (104) configured to be inserted into a reamed-out or broached portion of a bone (102) of a patient. The first implantable component can extend between a first end (110) and a second end (112). The first implantable component can also include a central body (114) extending from the first end of the first implantable component toward the second end of the first implantable component and one or more fins (116) attached to a periphery of the central body. The fins can extend radially outward the central body and extend from the first end of the first implantable component to the second end of the first implantable component. The central body and the one or more fins can be configured to be inserted into the reamed-out or broached portion of the bone of a patient from a superior approach.

### CLAIM OF PRIORITY

This application claims the benefit of U.S. Provisional Patent Application Ser. No. 63/428,284, filed on Nov. 28, 2022, the benefit of priority of which is claimed hereby, and which is incorporated by reference herein in its entirety.

### BACKGROUND

The present disclosure relates to an implant. More specifically, this disclosure relates to an implant configured to be inserted into a reamed-out or broached portion of a bone.

The shoulder joint is a complex joint with the scapula, clavicle, and humerus all coming together to enable a wide range of movement, at least in a properly functioning joint. In a properly functioning shoulder joint, the head of the humerus fits into a shallow socket in the scapula, typically referred to as the glenoid. Articulation of the shoulder joint involves movement of the humeral head in the glenoid, with the structure of the mating surfaces and surrounding tissues providing a wide range of motion.

The shoulder joint can undergo degenerative changes caused by various issues, such as rheumatoid arthritis, osteoarthritis, rotator cuff arthroplasty, avascular necrosis, or bone fracture. When severe joint damage occurs, and no other means of treatment is found to be effective, a total, partial, or reverse shoulder replacement or reconstruction may be necessary. Total shoulder replacements can involve a humeral prosthetic used to replace the natural humeral head. Total shoulder replacements will also typically include resurfacing of the glenoid with a prosthetic implant. The glenoid implant will generally include an articulating cup-shaped to receive the prosthetic humeral head. A reverse shoulder replacement (arthroplasty) involves a different set of humeral and glenoid replacement prosthetics. In a reverse shoulder replacement, the humeral component includes a cup-shaped articular surface attached to a stem implanted into the humerus, while a spherical glenoid component is used to provide an articular surface for the humeral cup.

### SUMMARY

The inventor of the present disclosure has recognized at least the following problems with shoulder implants generally: 1) the need to dislocate the humerus during shoulder replacement surgeries, 2) the need to detach the subscapular muscle of the rotator cuff during the insertion of an implant, and 3) complications reattaching the subscapular muscle after the implant is inserted. The following examples help address at least the above-mentioned issues with shoulder implants.

In an example, an implant can be configured to be implanted into a reamed-out or broached portion of a bone of a patient. The implant can extend between a first end and a second end. The implant can include a central body extending from the first end toward the second end. The implant can also include one or more fins attached to a periphery of the central body. The central body and the one or more fins can form a primary plane at the first end of the implant. The one or more fins can extend radially outward from the central body and extend from the first end of the central body to the second end of the implant. The central body and the one or more fins can be angled from the primary plane such that the implant can be inserted into the reamed-out or broached portion of the bone from a direction substantially parallel to a longitudinal axis of the bone of the patient.

In another example, a system configured to be implanted into a patient can include a first implantable component configured to be inserted into a reamed-out or broached portion of a bone of a patient. The first implantable component can extend between a first end and a second end. The first implantable component can also include a central body extending from the first end of the first implantable component toward the second end of the first implantable component and one or more fins attached to a periphery of the central body. The fins can extend radially outward from the central body and extend from the first end of the first implantable component to the second end of the first implantable component. The central body and the one or more fins can be configured to be inserted into the reamed-out or broached portion of the bone of a patient from a superior approach.

In yet another example, a method of performing arthroplasty on at least one of a glenoid or a humerus of a glenohumeral joint can include forming an entry incision in soft tissue superior and lateral to the glenohumeral joint. Forming a passage from the entry incision to the glenohumeral joint. Inserting a cutting instrument through the passage and cutting a natural humeral head of the humerus along an articular margin of the humeral head while the humeral head has no dislocation at all with respect to the glenoid and remains in its anatomical position. Inserting an implant through the passage and securing the implant relative to the humerus with the implant oriented such that a longitudinal axis of a central body of the implant extends substantially parallel to a longitudinal axis of the humerus. The method can also include maintaining both a subscapularis muscle and a supraspinatus muscle associated with the glenohumeral joint intact and completely connected to surrounding bone during an entirety of the arthroplasty.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an implant inserted in a humerus bone of a patient, in accordance with at least one example of the present disclosure.

FIG. 2 is a side view of an implant, in accordance with at least one example of the present disclosure.

FIG. 3 is a perspective view of a body of an implant, in accordance with at least one example of the present disclosure.

FIG. 4 is a cross-sectional, perspective view of the implant of FIG. 3 taken along line 4-4, in accordance with at least one example of the present disclosure.

FIG. 5 is a schematic flowchart showing a process of assembling and installing an implant, in accordance with at least one example of the present disclosure.

FIG. 6 is a perspective view of an example of an implant, in accordance with at least one example of the present disclosure.

FIG. 7 is a perspective view of an example of an implant, in accordance with at least one example of the present disclosure.

FIG. 8 is a flowchart detailing a method of inserting an implant, in accordance with at least one example of the present disclosure.

### DETAILED DESCRIPTION

The present disclosure relates to a system including a stemless implant configured to be implanted into a reamed-out or broached portion of a bone of a patient. More specifically, the present disclosure relates to a system including a stemless implant configured to be implanted into a reamed-out or broached portion of a bone of a patient from a superior approach. The superior approach has the benefit of enabling a bone to be resected, reamed-out, broached, and or prepared for the insertion of the implant, and for an implant to be able to be inserted into the bone without fully detaching, tearing, or cutting a subscapularis of the patient. Maintaining the subscapularis of the patient helps support the implant, reduce the recovery time for the patient, and improve functional outcome.

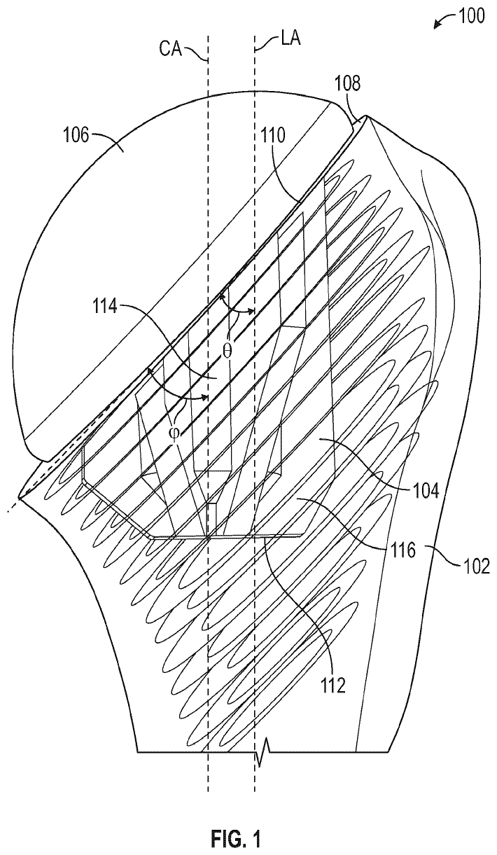

The implant can include a central body extending from a first end of the implant toward a second end of the implant and one or more fins (fins) attached to a periphery of the central body. The fins can form a primary plane at the first end of the implant. The central body and the fins can be angled from the primary plane such that the central body and the fins can be inserted into the reamed-out or broached portion of the bone from a direction substantially parallel with a longitudinal axis of the bone. The system including a stemless implant configured to be implanted into a reamed-out or broached portion of a bone of a patient will be discussed below with reference to FIGS. 1-8.

The above discussion is intended to provide an overview of subject matter of the present patent application. It is not intended to provide an exclusive or exhaustive explanation of the invention. The description below is included to provide further information about the present patent application.

FIG. 1 is a perspective view of a system 100 implanted into a patient. In examples, at least a portion of the system 100 can be inserted into a bone 102 of a patient. The system 100 can include a first implantable component, for example, an implant (hereinafter referred to as “implant 104”), and a second implantable component, for example, a prosthetic humeral head, or cup member, each configured to interact with a glenoid attachment (hereinafter referred to as “glenoid interface component 106”).

As shown in FIG. 1, the bone 102 can be a humerus bone. In another example, the bone 102 can be any other bone in a patient. The bone 102 can be an elongated body including a longitudinal axis LA. In examples, the bone 102 can be resected to define a resected surface 108. The bone 102 can be resected based on the anatomical features of the bone 102. Because the bone 102 can be resected based on anatomical features, the bone 102 can be resected before the bone 102 is reamed-out or broached.

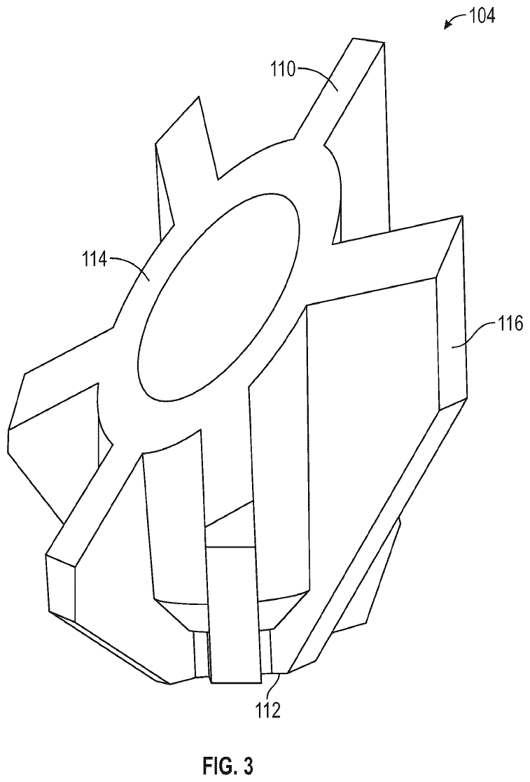

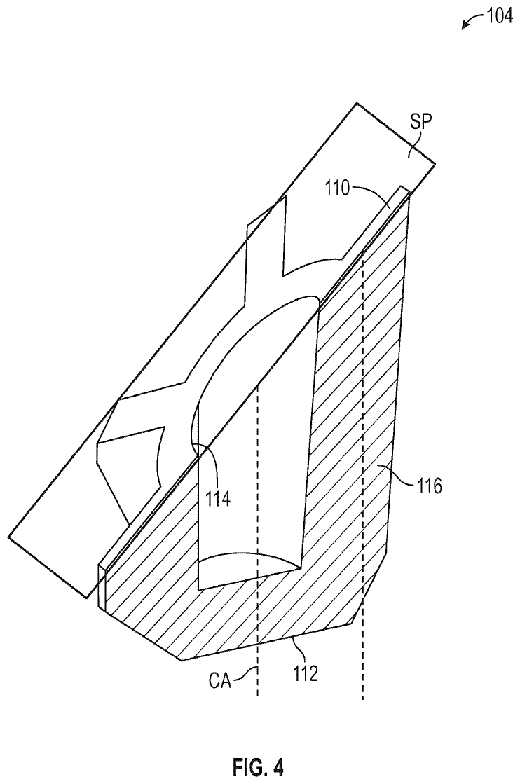

The implant 104 can be configured to be inserted into the bone 102 of a patient. The implant 104 extends between first end 110 and second end 112 along a central axis CA. The implant 104 can include a central body 114 and one or more fins (fins 116). As shown in FIG. 1, the central body 114 and the fins 116 can extend along the central axis CA from the first end 110 at an angle o. The central body 114, the fins 116, and the angle φ will be discussed below in more detail with reference to FIGS. 3 and 4.

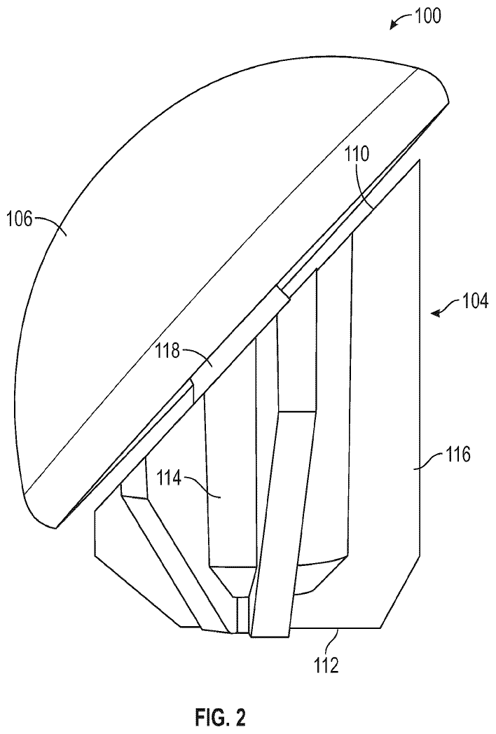

The glenoid interface component 106 can be configured to be attached to the implant 104. The glenoid interface component 106 can be a prosthetic humeral head, as shown in FIG. 1. In another example, the glenoid interface component 106 can be a humeral cup, or any other secondary attachment component configured to interact with another implant or part of a human body. The glenoid interface component 106 will be discussed in greater detail below with reference to FIGS. 2, 5, and 6.

The resected surface 108 can be configured to receive the bone 102 of the system 100. The resected surface 108 can be resected at an angle θ, which is an angle between the longitudinal axis LA and the resected surface 108 of the bone 102. The angle θ can vary based on the anatomical geometry of the bone 102. For example, the angle θ can be based on an articular margin of the humeral head of the bone 102. Because the implant 104 is configured to be inserted along the central axis CA, which can be substantially parallel to the longitudinal axis LA, the resected surface 108 can be less precise than usual shoulder implant surgeries. The decreased precision required for the resected surface 108 can save time and make the implant process more adjustable to the bone shape of the patient.

FIG. 2 is a side view of the system 100, which is configured to be implanted into a patient. The system 100 can include an attachment mechanism 118. The attachment mechanism 118 can be configured to attach the implant 104 and the glenoid interface component 106. In examples, the attachment mechanism 118 can be a standalone unit, or in other examples, the attachment mechanism 118 can be integral the implant 104 or the glenoid interface component 106, respectively. The attachment mechanism 118 can be configured to provide a gap between the implant 104 and the glenoid interface component 106 while the attachment mechanism 118 couples the implant 104 and the glenoid interface component 106. Because the attachment mechanism 118 creates the gap between the implant 104 and the glenoid interface component 106, the implant 104 can be completely within the bone 102 (FIG. 1) and the glenoid interface component 106 can be outside the bone 102 and in contact with the resected surface 108.

In the example of FIG. 2, the fins 116 can extend between the implant 104 and the glenoid interface component 106 in a direction that is substantially perpendicular to the resected surface 108.

The implant 104 will be discussed below with reference to FIGS. 3 and 4. FIG. 3 is a perspective view of the implant 104. FIG. 4 is a cross-sectional view of the implant 104 of FIG. 3 taken along line 4-4.

In the examples shown in FIGS. 3 and 4, the central body 114 can be hollow. When the central body 114 is hollow, the central body 114 can be configured to receive the attachment mechanism 118.

As shown in FIGS. 1-4, the fins 116 can have a shape that helps the implant 104 be inserted into the bone 102 in a direction that is substantially parallel to the longitudinal axis LA of the bone 102. For example, the fins 116 can extend from the first end 110 to the second end 112 along fin axis FA. The fin axis FA can be substantially parallel to the central axis CA. As shown in FIGS. 1-4, the second end 112 of the implant 104 can be horizontal, or, for example, create a plane (e.g., surface plane SP) that is substantially perpendicular to the central axis CA. In examples, the fins 116 can have a smaller horizontal cross-sectional area at the second end 112 and a maximum cross-sectional area at the first end 110. In another example, the fins 116 can have a minimum horizontal cross-sectional area at the second end 112 and can taper to a maximum cross-sectional area somewhere between the second end 112 and the first end 110, and then have a consistent cross-sectional area from the end of the taper to the first end 110. In yet another example, the fins 116 can have a minimum cross-sectional area at the second end 112 and can have a varying cross-sectional area while extending from the second end 112 to the first end 110, with a maximum cross-sectional area between the first end 110 and the second end 112 and a cross-sectional area between the minimum and the maximum at the first end 110. In another example, the fins 116 can be any shape that helps fill up the area within the bone 102 underneath the resected surface 108. The greater the area the fins 116, the greater dispersion of stresses acted upon the implant 104, and the more difficult the fins 116 can be to install within the bone 102 of a patient. In examples, the fins 116 can include a thickness that varies. For example, the fins 116 can have a minimum cross-sectional thickness at a distal end portion of the fins 116.

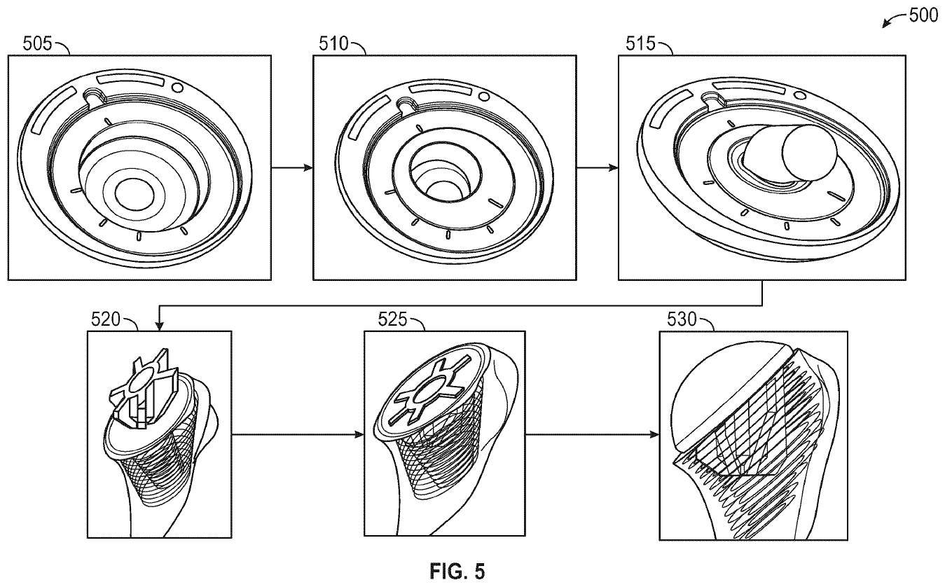

FIG. 5 is a schematic flowchart showing a process 500 of assembling and installing an implant, in accordance with at least one example of the present disclosure. The process 500 includes at least the following operations. The following operations are discussed for illustrative purposes only and are not intended to be the only operations or the only order in which the operations can be implemented. The following operations.

In an example, operation 505 can be to retrieve a prosthetic humeral head. The prosthetic humeral head can be inserted into a fixture, placed on a table, or held in the hand of a person. The prosthetic humeral head can be positioned such that a cavity is exposed.

In an example, operation 510 can be to install a ring adapter into the cavity of the prosthetic humeral head. As shown in FIG. 1, the ring adapter can include markings that can be used to position the ring adapter within the cavity of the prosthetic humeral head. In such an example, the prosthetic humeral head may also have markings that can be configured to help position the ring adapter within the cavity of the prosthetic humeral head.

In an example, operation 515 can be to install a stem adapter within a cavity of the ring adapter. In examples, the ring adapter, the stem adapter, or the ring adapter and the stem adapter can be referred to as an attachment mechanism. As shown in FIG. 5, the attachment mechanism (e.g., the attachment mechanism 118 from FIG. 2) can be attached to the prosthetic humeral head (e.g., the glenoid interface component 106 from FIGS. 1 and 2) and extend from the prosthetic humeral head in a direction that allows the prosthetic humeral head and the attachment mechanism to be inserted into the implant (e.g., the central body 114 of the implant 104 from FIGS. 1-4) from the superior approach.

In an example, operation 520 can include inserting the implant into a reamed-out or broached portion of the bone. As will be discussed elsewhere, to prepare the bone for insertion of the implant, the bone must first be resected and reamed out. As shown in FIG. 5, the implant can be inserted into the reamed-out or broached portion of a bone along the longitudinal axis of the bone.

In an example, operation 525 can be to insert the implant within the reamed-out or broached portion of the bone such that an exposed surface of the implant is aligned with the resected surface. The implant is fully seated when the exposed surface is aligned with the resected surface, and the implant can now be ready to receive the prosthetic humeral head.

In an example, operation 530 can be to insert the stem adapter into the implant such that the stem adapter is fully seated within the implant. As discussed above, and shown in FIG. 5, the stem adapter and the prosthetic humeral head can be installed from a direction that is along the longitudinal axis of the bone. This direction can also be referred to as from the superior approach.

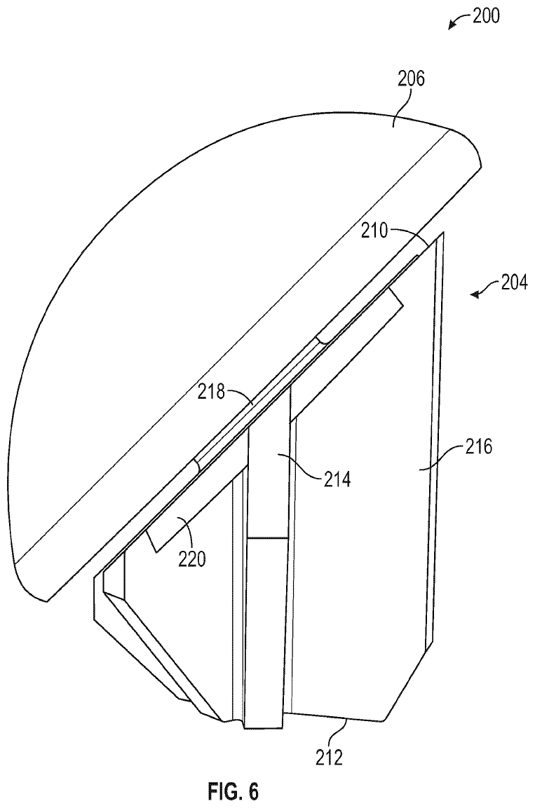

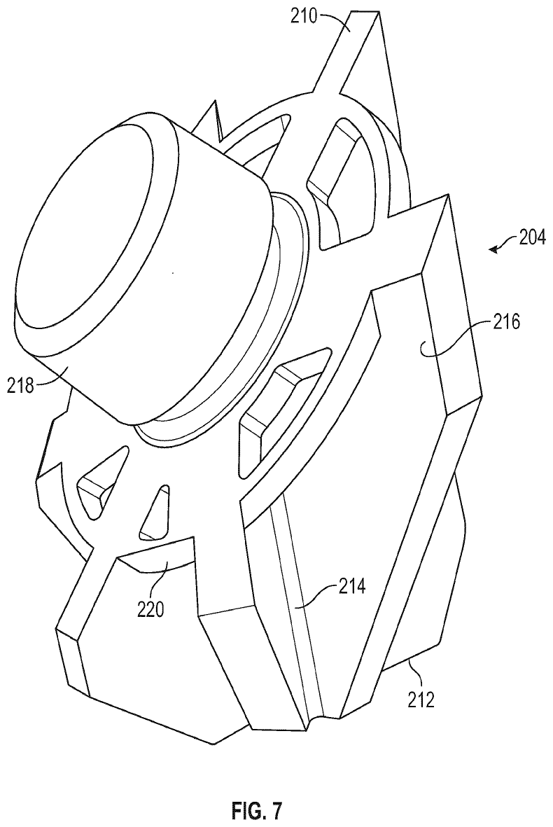

An alternative example of an implantable system will be discussed below with reference to FIGS. 6 and 7. FIG. 6 is a perspective view of an example of an implantable system (system 200). FIG. 7 is a perspective view of a portion of an example of the system 200. In the examples shown in FIGS. 6 and 7, the system 200 can include an implant 204, and a glenoid attachment interface 206. In examples, the implant 204 and the glenoid attachment interface 206 can be similar to the implant 104 and the glenoid interface component 106, respectively. The implant 204 can extend between a first end 210 and a second end 212 and include a central body 214, fins 216, and an attachment mechanism 218.

As shown in FIGS. 6 and 7, the attachment mechanism 218 can be a male trunnion attachment configured to attach to the glenoid attachment interface 206 of the system 200. Here, the attachment mechanism 218 can extend within the implant 204 and through the central body 214 to the second end 212 of the implant 204. In another example, the attachment mechanism 218 can extend within the implant 204 and through the central body 214 toward the second end 212 of the implant 204. As shown in FIGS. 6 and 7, the attachment mechanism 218 can extend from the second end 212 of the implant 204 in a direction that is substantially normal the implant 204. In another example, the attachment mechanism 218 can extend from the second end 212 of the implant 204 in a direction that is substantially aligned with the central body 214 of the implant 204. In such an example, the glenoid attachment interface 206 can be attached to the attachment mechanism 218 from the superior approach.

As shown in the example shown in FIGS. 6 and 7, the implant 204 can also include a collar 220. The collar 220 can extend around the first end 210 of the implant 204 and attach to each of the fins 216. The collar 220 can help support the fins 216 during the implantation of the implant 104 into a bone of a patient.

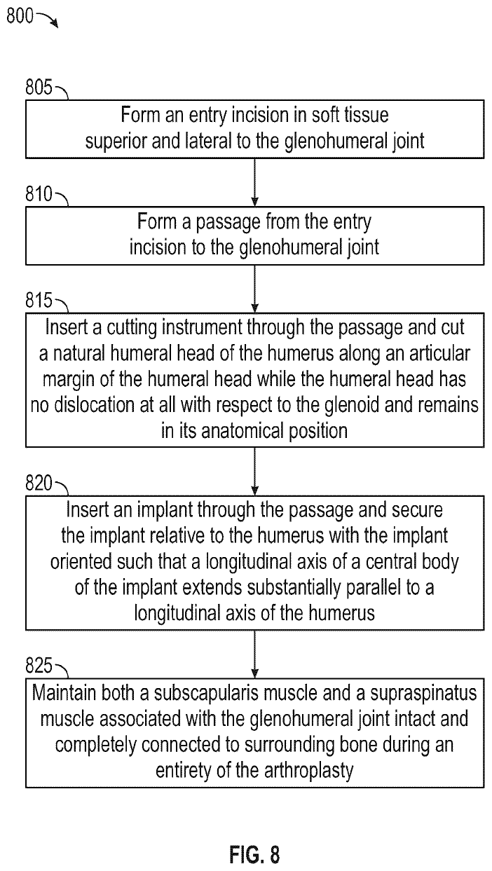

FIG. 8 illustrates a schematic view of the method 800, in accordance with at least one example of this disclosure. The method 800 can be a method of implanting a stemless implant from a superior approach. More specific examples of the method 800 are discussed below. The steps or operations of the method 800 are illustrated in a particular order for convenience and clarity; many of the discussed operations can be performed in a different sequence or in parallel without materially impacting other operations. The method 800 as discussed includes operations performed by multiple different actors, devices, and/or systems. It is understood that subsets of the operations discussed in the method 800 can be attributable to a single actor, device, or system could be considered a separate standalone process or method.

At operation 805, the method 800 can include forming an entry incision in soft tissue superior and lateral to the glenohumeral joint. Here, a surgeon can use a scalpel or any other knife to cut the skin and soft tissue (e.g., muscles) of the patient. The entry incision permits access to the shoulder area of a patient and enables the surgeon to pass various tools (e.g., endoscopes, scalpels, or any other tool) through the incision and into the interior of the patient.

It will be understood that the incision can be oriented in any appropriate direction such as anterior to posterior, which is generally parallel to a sagittal plane. In this regard, the incision is about 5 cm in length. Alternatively, or in addition thereto, a superior-inferior incision, which is generally along the coronal plan, can be made. The skin incision can be made parallel with Langer's lines at the superior aspect of the shoulder, just even with the lateral border of acromion. The incision can also be medialized slightly. The incision can be any appropriate length, and can depend upon surgeon preference, patient type, prosthetics to be used, or other indications. Nevertheless, the incision can be about 3 cm (about 1 in) to about 20 cm (about 8 in) in length, such as about 7.5 cm (about 3 in) to about 10 cm (4 in). It will be understood that the incision through the skin may be shorter than the area opened in the muscle. The incision can be used to achieve access to the muscle that is around the various portions of the anatomy that are selected to be resected, including the natural humerus and the natural glenoid. The incision can be used to obtain access to a deltoid muscle.

A retractor can be used to open the incision. The retractor can be any appropriate retractor such as a Gelpi Style Retractor. It will be understood that the retractor may also be used to retract the soft tissue, such as the muscles surrounding the glenohumeral joint, including the deltoid muscle, but the Gelpi Style Retractor can also be used to expand the incision to gain access to the muscle. The retractor can be used to retract or position the deep tissue that is generally near the glenohumeral joints.

At operation 810, the method 800 can include forming a passage from the entry incision to the glenohumeral joint. Here, the surgeon can use a tool (e.g., an endoscope, scalpel, or any combination of tools) to create a passage from the entry incision to the glenohumeral joint of the patient. The passage permits various tools, and surgical debris to pass from the incision to the glenohumeral joint, and from the glenohumeral joint to the incision.

The passage through the deltoid muscle can achieve access to various deeper soft tissue portions, such as the sub-deltoid bursa and the subacromial bursa without damaging the rotator cuff. Further, various other deep soft tissue can be incised or moved to access the capsule surrounding the shoulder or glenohumeral joint. After moving or incising all of these portions, access to the natural humerus or the natural humeral head can be achieved.

The retractor can be used to hold the various soft tissues portions open, such as the cuff interval, capsule and the like. It will be understood that the retractor may be any appropriate retractor and may include a scissor retractor or the like. Various other soft tissue portions may be near the capsule and may also be incised or resected. For example, the bicep tendon that interconnects to a portion or near the natural humeral head can be resected or can be moved, if already detached, to achieve better access to the natural humeral head. Further, access to the natural glenoid can also be seen once the soft tissue has been incised.

Although the incision on the shoulder or near the glenohumeral joint allows access to the deltoid muscle and access to the capsule and soft tissue surrounding the glenohumeral joints, various muscles and ligaments need not be resected or incised when obtaining access to the glenohumeral joint according to the process discussed herein. For example, the subscapularis muscle and the ligaments attaching it to the portions of the glenohumeral joint need not be incised when obtaining access to the glenohumeral joint according to embodiments of the teachings herein. The subscapularis muscle can be left intact, or partially intact because it is generally anterior from the approach described. Also, the supraspinatus can remain intact or partially intact, as may all the rotator cuff muscles. Rather the passage can be formed by separating the cuff interval rather than detaching or incising various soft tissue portions. Further, the natural humeral head need not be substantially dislocated or dislocated at all from the glenohumeral joint according to various embodiments. Rather, the natural humeral head can be moved to allow access to various portions of the anatomy, however, major dislocation of the natural humeral head from the natural glenoid is not necessary. The humerus can be left in its generally anatomical position or retracted at any appropriate distance, such as about 2 cm to about 8 cm.

The soft tissue over the biceps laterally is sharply dissected off the humerus down to the top of the subscapularis tendon, but the tendon can be left undisturbed. The supraspinatus may be stripped back off the anterior portion of the greater tuberosity for a distance of about 5 mm to about 10 mm to further enhance the exposure. More than about 1 cm may not be detached, and the basic integrity of the tendon can remain. This exposure of the rotator interval typically gives about a 1.5 cm to about 2 cm gap at the lateral edge, without disrupting the rotator cuff mechanism. The Gelpi retractor can be moved from the deltoid muscle to the rotator interval, which can provide greater exposure of the glenohumeral joint for instrumentation and implants.

At operation 815, the method 800 can include inserting a cutting instrument through the passage and cutting a natural humeral head of the humerus along an articular margin of the humeral head while the humeral head has no dislocation at all with respect to the glenoid and remains in its anatomical position. For example, the surgeon may use a bone saw to cut the articular margin of the humeral head. Further, the cutting instrument can be a reamer used to ream out a portion of the bone to prepare the bone to receive the implant. Further, the cutting instrument can be a bore used to bore out the bone in preparation such that the bone can receive the implant.

Once access has been obtained to the glenohumeral joint, various instruments according to various embodiments can be used in the procedure. It will be understood that according to various embodiments, any, all, or none of the instruments may be used in a procedure. For example, the glenohumeral joint can be reamed into the natural humerus near the natural humeral head. Humeral reaming can occur from the superior, lateral humeral head. The entrance to the natural humeral head can be just underneath the previous location (i.e., the natural location) of the biceps tendon. The arm can be extended slightly, and the elbow can be placed against the patient's side to bring the top of the natural humeral head forward and allow the reamer to pass the front of the acromion. This can allow the natural humeral head to be retracted but remain substantially or completely undislocated. This can reduce trauma in the surrounding soft tissue. The superior approach allows easy centering of the reamer in the humeral head and proximal shaft and decreases the initial incidence of varus stem placement or eccentric head utilization.

Once the natural humeral head has been resected, thus forming the resected natural humeral head, the natural glenoid can be reamed with a reamer. It will be understood that the natural glenoid may be first prepared with the various procedures according to those commonly known in the art. As discussed above, various connecting portions can be positioned at a superior or top portion of the sizer or other instrument to assist in achieving the superior approach described herein. The reamer can be interconnected with a reamer shaft and a drill motor. This allows the reamer to be rotated relative to the natural glenoid to form the glenoid into a selected shape and orientation. The natural glenoid may need to be shaped to allow for implantation of a selected glenoid implant. Nevertheless, it will be understood that the natural glenoid need not necessarily be resected and may articulate with an implant positioned in the resected natural humerus.

Regardless, the reamer can be positioned relative to the natural glenoid, and the shaft extends through the incision to allow for interconnection with the drill motor. After a selected period of reaming, the natural glenoid can be prepared for implantation of a glenoid implant. As discussed above, the glenoid prosthetic can be positioned relative to the reamed natural glenoid to assist in further glenoid preparation.

Once the various bones of the anatomy have been resected, including the natural glenoid and the natural humerus, the various implants can be implanted. The natural humerus may need to be further prepared, such as broaching the IM canal of the natural humerus. Therefore, a broach may be provided and used to broach a selected portion of the proximal portion of the natural humerus. Various sizes of broaches may be used to progressively enlarge the broached area of the natural humerus, as is generally known in the art.

At operation 820, the method 800 can include inserting an implant through the passage and securing the implant relative to the humerus with the implant oriented such that a longitudinal axis of a central body of the implant extends substantially parallel to a longitudinal axis of the humerus.

After inserting the implants (e.g., the implant 104 from FIG. 1 or the implant 204 from FIG. 2) into the medullary canal using impaction, the humeral head (e.g., the glenoid interface component 106 from FIG. 1 or the glenoid attachment interface 206 from FIG. 2) can be coupled to the locking taper (e.g., the attachment mechanism 118 from FIG. 1 or the attachment mechanism 218 from FIG. 2) formed on the proximal end. An impact force is applied to the stem in a direction of the central stem axis CA.

At operation 825, the method 800 can include maintaining both a subscapularis muscle and a supraspinatus muscle associated with the glenohumeral joint intact and completely connected to surrounding bone during an entirety of the arthroplasty. Here, because the implant permits insertion from the superior approach, the implant system can be implanted into the body of a patient without dislocating the humerus or tearing the subscapularis or the supraspinatus muscles. As such, a patient can have a faster recovery with an increased predictability of success.

Following implantation, the soft tissue balance can again be assessed, and then the split in the rotator interval can be closed. The deltoid can be repaired back to the acromion. Subcutaneous tissues and skin can then be closed per the surgeon's usual routine.

Various portions described herein can be provided in a system. The system can include any appropriate portions that can be used in a selected procedure, such as a glenoid and/or humeral procedure, and can include a plurality of the broaches, a plurality of the humeral implants, a plurality of humeral heads, a plurality of the glenoid implants, and any other appropriate portion. Therefore, the system can be used for a plurality of procedures and need not be customized for a particular procedure or patient. Further, the system can include a plurality of portions that allow it to be used in several procedures for many differing anatomies, sizes, and the like. Further, various other portions, such as the reamer, the glenoid template, or other appropriate portions can be provided for a plurality of different patients.

### NOTES AND EXAMPLES

The following, non-limiting examples, detail certain aspects of the present subject matter to solve the challenges and provide the benefits discussed herein, among others.

Example 1 is an implant configured to be implanted into a reamed-out or broached portion of a bone of a patient, the implant extending between a first end and a second end, the implant comprising: a central body extending from the first end toward the second end; and one or more fins attached to a periphery of the central body such that the central body and the one or more fins form a primary plane at the first end of the implant, the one or more fins extend radially outward from the central body and extend from the first end of the central body to the second end of the implant, the central body and the one or more fins angled from the primary plane such that the implant can be inserted into the reamed-out or broached portion of the bone from a direction substantially parallel a longitudinal axis of the bone of the patient.

In Example 2, the subject matter of Example 1 includes, wherein the central body further comprises: an attachment mechanism configured to attach the implant to a second implant component.

In Example 3, the subject matter of Example 2 includes, wherein the attachment mechanism is a male tapered trunnion extending from the first end of the central body at an extension angle.

In Example 4, the subject matter of Example 3 includes, wherein the extension angle and the angle between the primary plane and the central body and the one or more fins are adjacent pairs.

In Example 5, the subject matter of Examples 3-4 includes, wherein the extension angle is normal the primary plane.

In Example 6, the subject matter of Examples 2-5 includes, wherein the attachment mechanism is a female tapered trunnion extending within the central body from the first end of the implant toward the second end of the implant.

In Example 7, the subject matter of Example 6 includes, wherein the female tapered trunnion extends within the central body at an angle substantially equal to the angle between the primary plane and the central body and the one or more fins.

In Example 8, the subject matter of Examples 6-7 includes, the female tapered trunnion extends within the central body substantially normal to the primary plane.

Example 9 is a system configured to be implanted into a patient, the system comprising: a first implantable component configured to be inserted into a reamed-out or broached portion of a bone of a patient, the first implantable component extending between a first end and a second end, the first implantable component comprising: a central body extending from the first end of the first implantable component toward the second end of the first implantable component; and one or more fins attached to a periphery of the central body, the one or more fins extend radially outward the central body and extend from the first end of the first implantable component to the second end of the first implantable component, the central body and the one or more fins are configured to be inserted into the reamed-out or broached portion of the bone of a patient from a superior approach.

In Example 10, the subject matter of Example 9 includes, wherein the system further comprises an attachment mechanism configured to attach the first implantable component to a second implantable component.

In Example 11, the subject matter of Example 10 includes, wherein the attachment mechanism is a male tapered trunnion extending from the first end of the first implantable component.

In Example 12, the subject matter of Example 11 includes, wherein the second implantable component is a prosthetic humeral head configured to be attached to the first implantable component via the male tapered trunnion.

In Example 13, the subject matter of Example 12 includes, wherein the male tapered trunnion extends from the first end of the first implantable component such that the prosthetic humeral head can be impacted onto the first implantable component from the superior approach.

In Example 14, the subject matter of Examples 11-13 includes, wherein the second implantable component is a cup member configured to be attached to the first implantable component via the male tapered trunnion.

In Example 15, the subject matter of Example 14 includes, wherein the male tapered trunnion extends from the first end of the first implantable component such that the cup member can be impacted onto the first implantable component from the superior approach.

In Example 16, the subject matter of Examples 10-15 includes, wherein the attachment mechanism is a female tapered trunnion extending from the first end of the first implantable component within the central body.

In Example 17, the subject matter of Example 16 includes, wherein the second implantable component is a prosthetic humeral head configured to be attached to the first implantable component via the female tapered trunnion, wherein the female tapered trunnion extends within the central body of the first implantable component such that the prosthetic humeral head can be impacted onto the first implantable component from the superior approach.

In Example 18, the subject matter of Examples 16-17 includes, wherein the second implantable component is a cup member configured to be attached to the first implantable component via the female tapered trunnion, wherein the female tapered trunnion extends within the central body of the first implantable component such that the cup member can be impacted onto the first implantable component from the superior approach.

Example 19 is a method of performing arthroplasty on at least one of a glenoid or a humerus of a glenohumeral joint comprising: forming an entry incision in soft tissue superior and lateral to the glenohumeral joint; forming a passage from the entry incision to the glenohumeral joint; inserting a cutting instrument through the passage and cutting a natural humeral head of the humerus along an articular margin of the humeral head while the humeral head has no dislocation at all with respect to the glenoid and remains in its anatomical position; inserting an implant through the passage and securing the implant relative to the humerus with the implant oriented such that a longitudinal axis of a central body of the implant extends substantially parallel to a longitudinal axis of the humerus; and maintaining both a subscapularis muscle and a supraspinatus muscle associated with the glenohumeral joint intact and completely connected to surrounding bone during an entirety of the arthroplasty.

In Example 20, the subject matter of Example 19 includes, inserting a reamer through the entry incision; reaming the glenoid with the reamer; inserting a glenoid implant through the passage; and securing the glenoid implant to the glenoid.

Example 21 is an apparatus comprising means to implement of any of Examples 1-20.

Example 22 is a system to implement of any of Examples 1-20.

Example 23 is a method to implement of any of Examples 1-20.

In Example 24, the apparatuses or method of any one or any combination of Examples 1-24 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An implant configured to be implanted into a reamed-out or broached portion of a bone of a patient, the implant extending between a first end and a second end, the implant comprising:
a central body extending from the first end toward the second end; and
one or more fins attached to a periphery of the central body such that the central body and the one or more fins form a primary plane at the first end of the implant, the one or more fins extend radially outward from the central body and extend from the first end of the implant toward the second end of the implant, the central body and the one or more fins orientated at a major angle relative to the primary plane such that the implant can be inserted into the reamed-out or broached portion of the bone from a direction substantially parallel a longitudinal axis of the bone of the patient.

2. The implant of claim 1, wherein the central body further comprises:
an attachment mechanism configured to attach the implant to a second implant component.

3. The implant of claim 2, wherein the attachment mechanism is a male tapered trunnion extending from the first end of the implant at an extension angle.

4. The implant of claim 3, wherein the extension angle and the major angle are adjacent pairs.

5. The implant of claim 3, wherein the extension angle is normal to the primary plane.

6. The implant of claim 2, wherein the attachment mechanism is a female tapered trunnion extending within the central body from the first end of the implant toward the second end of the implant.

7. The implant of claim 6, wherein the female tapered trunnion extends within the central body at an angle substantially equal to the major angle.

8. The implant of claim 6, wherein the female tapered trunnion extends within the central body substantially normal to the primary plane.

9. A system configured to be implanted into a patient, the system comprising:
a first implantable component configured to be inserted into a reamed-out or broached portion of a bone of a patient, the first implantable component extending between a first end and a second end, the first implantable component comprising:
a central body extending from the first end of the first implantable component toward the second end of the first implantable component; and
one or more fins attached to a periphery of the central body, the one or more fins extend radially outward from the central body and extend from the first end of the first implantable component to the second end of the first implantable component, the central body and the one or more fins are oriented at a major angle relative to the first end of the first implantable component, the major angle enables insertion of the first implantable component into the reamed-out or broached portion of the bone of the patient from a superior approach.

10. The system of claim 9, wherein the system further comprises an attachment mechanism configured to attach the first implantable component to a second implantable component.

11. The system of claim 10, wherein the attachment mechanism is a male tapered trunnion extending from the first end of the first implantable component.

12. The system of claim 11, wherein the second implantable component is a prosthetic humeral head configured to be attached to the first implantable component via the male tapered trunnion.

13. The system of claim 12, wherein the male tapered trunnion extends from the first end of the first implantable component at an angle that is substantially parallel to the major angle such that the prosthetic humeral head can be impacted onto the first implantable component from the superior approach.

14. The system of claim 11, wherein the second implantable component is a cup member configured to be attached to the first implantable component via the male tapered trunnion.

15. The system of claim 14, wherein the male tapered trunnion extends from the first end of the first implantable component such that the cup member can be impacted onto the first implantable component from the superior approach.

16. The system of claim 10, wherein the attachment mechanism is a female tapered trunnion extending from the first end of the first implantable component within the central body.

17. The system of claim 16, wherein the second implantable component is a prosthetic humeral head configured to be attached to the first implantable component via the female tapered trunnion, wherein the female tapered trunnion extends within the central body of the first implantable component such that the prosthetic humeral head can be impacted onto the first implantable component from the superior approach.

18. The system of claim 16, wherein the second implantable component is a cup member configured to be attached to the first implantable component via the female tapered trunnion, wherein the female tapered trunnion extends within the central body of the first implantable component such that the cup member can be impacted onto the first implantable component from the superior approach.

19. A method of performing arthroplasty on at least one of a glenoid or a humerus of a glenohumeral joint comprising:
forming an entry incision in soft tissue superior and lateral to the glenohumeral joint;
forming a passage from the entry incision to the glenohumeral joint;
inserting a cutting instrument through the passage and cutting a natural humeral head of the humerus along an articular margin of the natural humeral head while the natural humeral head has no dislocation at all with respect to the glenoid and remains in its anatomical position;
inserting an implant through the passage and securing the implant relative to the humerus with the implant oriented such that a longitudinal axis of a central body of the implant extends substantially parallel to a longitudinal axis of the humerus; and
maintaining both a subscapularis muscle and a supraspinatus muscle associated with the glenohumeral joint intact and connected to surrounding bone during an entirety of the arthroplasty.

20. The method of claim 19, further comprising:
inserting a reamer through the entry incision;
reaming the glenoid with the reamer;
inserting a glenoid implant through the passage; and
securing the glenoid implant to the glenoid.
